(19) United States
(12) Patent Application Publication
(10) Pub. No.: US 2023/0371884 A1
(43) Pub. Date: Nov. 23, 2023

(54) GARMENT INCLUDING FETAL MONITORING SENSORS, AND SYSTEMS AND METHODS OF USE

(71) Applicant: Rubi Life, LLC, South Jordan, UT (US)

(72) Inventors: Anton E. Bowden, Lindon, UT (US); Kyle Pulsipher, Provo, UT (US); Dillon Despain, Provo, UT (US)

(21) Appl. No.: 18/246,961

(22) PCT Filed: Sep. 28, 2021
(86) PCT No.: PCT/US2021/052368

§ 371 Date: Mar. 28, 2023

(30) Foreign Application Priority Data
Oct. 1, 2020 (US) 63086266

(51) Int. Cl. A61B 5/00; A61B 5/11

(52) U.S. Cl. CPC A61B 5/4362; A61B 5/6804; A61B 5/11; A61B 2562/0261; A61B 2562/046

(57) ABSTRACT

A fetal monitoring system includes a garment configured to be worn by a pregnant woman a plurality of sensors positioned on the garment to be over the abdomen of the pregnant woman. The plurality of sensors are positioned or positionable in a predetermined arrangement on the garment and configured to sense or detect movement of at least one of the pregnant women or an in utero fetus within the abdomen of the pregnant women. The fetal monitoring system also includes one or more conductive wires connecting each sensor of the plurality of sensors, a communication module connected to at least one of the one or more conductive wires and configured to transmit data from the plurality of sensors, and a controller configured communicate with the communication module and receive the data from the plurality of sensors transmitted by the communication module.

### CROSS-REFERENCE TO RELATED APPLICATIONS

This application claims priority to U.S. Provisional Patent Application No. 63/086,266 filed on Oct. 1, 2020, the disclosure of which is incorporated herein, in its entirety, by this reference.

### BACKGROUND

Detection of movement of in utero fetuses may help indicate the health of the fetuses. Moreover, detection of movement of a pregnant woman may help indicate the health of the pregnant woman and/or potential emergencies involving the pregnant woman. Thus, users and manufacturers continue to seek new and improved systems for monitoring movement of fetuses and/or pregnant women.

### SUMMARY

Embodiments disclosed herein are garments including fetal monitoring sensors, and related systems and methods. In an embodiment, a fetal monitoring system includes a garment configured to be worn by a pregnant woman, a plurality of sensors, one or more conductive wires, a communication module, and a controller. The plurality of sensors may be positioned or positionable in a predetermined arrangement on the garment to be over the abdomen of the pregnant woman and configured to sense or detect movement of at least one of the pregnant women or an in utero fetus within the abdomen of the pregnant women. The predetermined arrangement includes a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region at one or more second distances greater than the one or more first distances. The one or more conductive wires connect each sensor of the plurality of sensors. The communication module is connected to at least one of the one or more conductive wires and configured to transmit data from the plurality of sensors. The controller is configured communicate with the communication module and receive the data from the plurality of sensors transmitted by the communication module.

In an embodiment, a garment for use in a fetal monitoring system is described. The garment includes a region positioned to cover the abdomen of a pregnant women. The garment also includes a plurality of sensors positioned or positionable in a predetermined arrangement on the region of the garment to be over the abdomen of the pregnant woman and configured to sense or detect movement of at least one of the pregnant women or an in utero fetus within the abdomen of the pregnant women. The predetermined arrangement of the plurality of sensors includes a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region at one or more second distances greater than the one or more first distances.

In an embodiment, a method of monitoring an in utero fetus is described. The method includes positioning a garment on a pregnant woman with a plurality of sensors positioned in a predetermined arrangement on the garment over the abdomen of the pregnant woman. The predetermined arrangement includes a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region at one or more second distances greater than the one or more first distances. The method also includes an act of detecting, with at least one of the plurality of sensors, movement of at least one of the pregnant women or an in utero fetus within the abdomen of the pregnant women. The method also includes transmitting, with a communication module at least proximate to the garment, data associated with the movement detected by the at least one of the plurality of sensors. The method also includes receiving, with a controller, the data transmitted by the communication module.

In an embodiment, a method of forming a garment for a fetal monitoring system is described. The method includes positioning a plurality of sensors on a region of the garment in a predetermined arrangement to be over that abdomen of a pregnant woman when worn by the pregnant woman. The plurality of sensors are configured to sense or detect movement of at least one of the pregnant women or an in utero fetus within the abdomen of the pregnant women. The predetermined arrangement includes a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region at one or more second distances greater than the one or more first distances. The method also includes connecting the plurality of sensors with one or more conductive wires.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a front view of a garment including fetal monitoring sensors, according to an embodiment.

FIG. 2 is a front view of a garment including fetal monitoring sensors, according to an embodiment.

FIG. 3 is a front view of a garment including fetal monitoring sensors, according to an embodiment.

FIG. 4 is a front view of a garment including fetal monitoring sensors, according to an embodiment.

FIG. 5 is a schematic of a controller, according to an embodiment.

FIG. 6 is a flow diagram of a method of monitoring an in utero fetus, according to an embodiment

FIG. 7 is a flow diagram of a method of forming a garment for a fetal monitoring system, according to an embodiment.

### DETAILED DESCRIPTION

Embodiments disclosed herein include garments having fetal monitoring sensors, and systems and methods of use. When the garment is worn by a pregnant woman, the fetal monitoring sensors are configured to sense or detect various movements that may be determined to be movement of an in utero fetus. The fetal monitoring sensors on the garment also may distinguish different types of fetal movements and/or distinguish fetal movements from movements of the pregnant woman.

Embodiments of garments for use in a fetal monitoring system are described herein. The garments may include a region of the garment that is positioned on the garment to cover the abdomen of a pregnant women. The garment also may include a plurality of sensors positioned or positionable in a predetermined arrangement on the region of the garment to be over the abdomen of the pregnant woman. The plurality of sensors are configured to sense or detect movement of at least one of the pregnant women or an in utero fetus within the abdomen of the pregnant women. The predetermined arrangement of the plurality of sensors may include a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region at one or more second distances greater than the one or more first distances. The garment also may include one or more conductive wires connecting each sensor of the plurality of sensors. The garment may be part of a fetal monitoring system, according to an embodiment. In these and other embodiments, the fetal monitoring system may include a communication module that is connected to at least one of the one or more conductive wires and configured to transmit data from the plurality of sensors. The fetal monitoring system also may include a controller configured communicate with the communication module and receive the data from the plurality of sensors transmitted by the communication module.

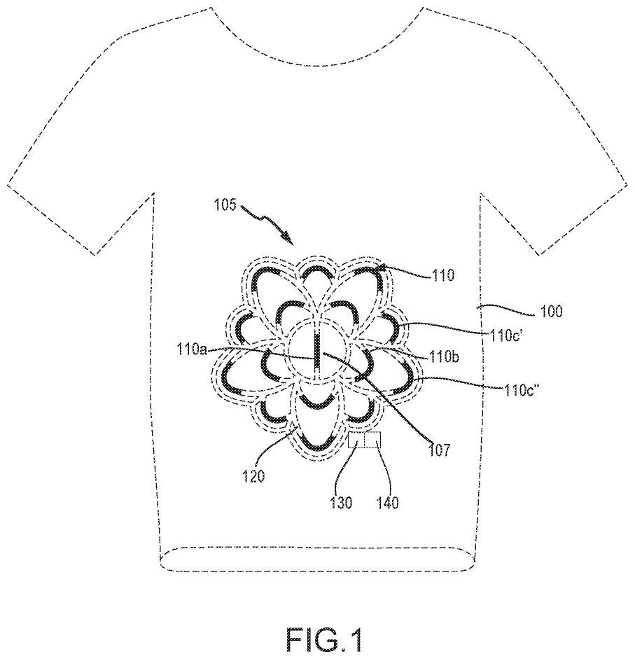

FIG. 1 is a front view of a garment 100 including a plurality of fetal monitoring sensors 110 positioned in a predetermined arrangement 105 on the garment 100 to be positioned over the abdomen of an individual wearing the garment 100, such as a pregnant woman, according to an embodiment. The predetermined arrangement 105 of the plurality of sensors 110 at least a central sensor 110a of the plurality of sensors 110 positioned in a central region 107 of the predetermined arrangement and multiple sensors of the plurality of sensors 110 radially arranged around the central sensor 110a. The plurality of sensors 110 are configured to sense or detect various movements of an in utero fetus in the pregnant women wearing the garment 100. The plurality of sensors 110 also may be configured to sense or detect various movements of the individual, such as the pregnant woman, wearing the garment 100. The garment 100 may include any garment configured to be worn by a pregnant woman with the plurality of sensors 110 positioned over the abdomen of the pregnant woman. For example, the garment 100 may include a short sleeve t-shirt, a long sleeve t-shirt, a button up shirt, an apron, a jacket, a dress, high-waist pants or tights, a tubular wrap or sleeve, or any combination thereof.

The plurality of sensors 110 may include any sensor configured to sense or detect various movements of the in utero fetus and/or the individual wearing the garment 100. In some embodiments, each of the plurality of sensors 110 include a plurality of strain gauges. More particularly, the plurality of sensors 110 may include strain gauges screen printed onto the garment 100. Screen printing ink, for example, may be blended with metal nano-particles to form a composite material that is screen printed on the garment 100 in a predetermined pattern to be the plurality of sensors 110. The metal nano-particles may include nickel coated carbon fibers and nickel nanostrands, and the screen printing ink may include a silicone ink base. In some embodiments, the composite material of screen printing ink and metal nano-particles includes greater than about 12% saturation of the metal nano-particles. In some embodiments, the composite material includes at least about 5% saturation of the metal nano-particles, at least about 10% saturation of the metal nano-particles, at least about 15% saturation of the metal nano-particles, at least about 20% saturation of the metal nano-particles, at least about 25% saturation of the metal nano-particles, at least about 30% saturation of the metal nano-particles, about 5% to about 25% saturation of the metal nano-particles, about 5% to about 10% saturation of the metal nano-particles, about 10% to about 15% saturation of the metal nano-particles, about 15% to about 20% saturation of the metal nano-particles, about 20% to about 25% saturation of the metal nano-particles, or about 20% to about 25% saturation of the metal nano-particles.

The composite material also may include about 5 wt % to about 8 wt % of catalyst. In some embodiments, the composite material may include about 2 wt % to about 16 wt % of catalyst, about 2 wt % to about 9 wt % of catalyst, about 9 wt % to about 16 wt % of catalyst, about 4 wt % to about 9 wt % of catalyst, about 3 wt % to about 10 wt % of catalyst, about 5 wt % to about 7 wt %, about 6 wt % to about 8 wt % of catalyst, about 4 wt % of catalyst, about 5 wt % of catalyst, about 6 wt % of catalyst, about 7 wt % of catalyst, about 8 wt % of catalyst, about 9 wt % of catalyst, or about 10 wt % of catalyst.

When printed on the garment 100, the plurality of sensors 110 may have a thickness of less than about 1.0 mm, such as about 0.75 mm, about 0.6 mm, about 0.5 mm, about 0.4 mm, about 0.3 mm, less than about 0.3 mm, about 0.1 mm to about 1 mm, about 0.1 mm to about 0.5 mm, about 0.5 mm to about 1.0 mm, about 0.1 mm to about 0.3 mm, about 0.3 mm to about 0.5 mm, about 0.5 mm to about 0.7 mm, or about 0.7 mm to about 0.9 mm. In some embodiments, the plurality of sensors 110 may include strain gauges secured to the garment 100 with adhesive, stitching, ultrasonic welding, or combinations thereof. Other sensors and positioning of sensors are disclosed in U.S. Patent Application No. 62/844,869, the disclosure of which is incorporated herein by reference in its entirety.

In some embodiments, at least some of the plurality of sensors 110 may be generally arched and concave relative to a central region 107 of the predetermined arrangement 105. In some embodiments, one or more of the plurality of sensors 110 may be generally straight. In some embodiments, the plurality of sensors 110 may include (1) a central sensor 110a that is generally straight and positioned at least proximate to the central region 107 of the predetermined arrangement and (2) multiple sensors 110b, 110c that are generally arched and concave relative to the central region 107. In some embodiments, all of the plurality of sensors 110 are generally straight.

The plurality of sensors 110 may be positioned on the garment 100 in predetermined positions (e.g., the predetermined arrangement 105) effective to sense movement of the fetus and/or the individual wearing the garment 100. The predetermined arrangement 105 of the plurality of sensors 110 provides more diagnostic information regarding fetal and/or maternal movement, breathing, and/or activity than would be conveyed by individual sensors alone. The relative positioning of the plurality of sensors 110 described herein allows for more effective diagnostic information regarding the fetus and the mother. FIG. 1 shows the garment 100 having the sensors 110 positioned in a first arrangement 105, according to an embodiment. In some embodiments, the plurality of sensors 110 include a central sensor 110a and multiple sensors radially arranged around the central sensor 110a. The multiple sensors radially arranged around the central sensor 110a may include a first portion 110b of sensors positioned in a first array around the central sensor 110a and a second portion 110c of sensors positioned in a second array around the first portion of the multiple sensors (and the central sensor 110a). Each sensor of the first portion 110b of sensors may be positioned closer to the central sensor 110a than each sensor of the second portion 110c of sensors. For example, the first portion 110b of the multiple sensors may be radially arranged around the central sensor 110a at one or more first distances and the second portion of sensors 110c of the multiple sensors may be radially arranged around the central sensor 110a at one or more second distances greater than the one or more first distances.

In some embodiments, the first portion 110b of sensors are radially arranged around the central sensor 110a (or the central region 107) and positioned equidistance from adjacent sensors of the first portion 110b of sensors. Accordingly, each sensor of the first portion 110b of sensors may be angled at equal angles between adjacent sensors of the first portion 110b of sensors relative to the central region 107. In the garment 100, the first portion 110b of sensors includes five sensors positioned in an array around the central sensor 110a. The five sensors of the first portion 110b of sensors may be positioned approximately 72° between adjacent sensors of the first portion of sensors relative to the central region 107. In some embodiments, the first portion 110b of sensors may include three, four, five, six, seven or more sensors and may be positioned substantially equidistance from adjacent sensors of the first portion 110b of sensors. The angle between adjacent sensors in the first portion 110b of sensors relative to the central region may be about 120° when the first portion 110b includes three sensors, about 90° when the first portion 110b includes four sensors, about 60° when the first portion 110b includes six sensors, about 51° when the first portion 110b includes seven sensors, and so on.

In some embodiments, the first portion 110b and the second portion 110c of sensors are positioned such that at least one sensor of the second portion 110c is positioned circumferentially between two sensors of the first portion 110b of sensors or radially spaced and between two adjacent sensors of the first portion 110b of sensors. In some embodiments, the second portion 110b of sensors may include a first group 110c′ of sensors and a second group 110c″ of sensors. The first group 110c′ of sensors may be positioned at a substantially equal first distance from the central sensor 110a and substantially equidistance from adjacent sensors of the first group of sensors. Each sensor of the first group 110c′ of sensors may be positioned circumferentially between two sensors of the first portion 110b of sensors. For example, a theoretical line extending between a sensor of the first group 110c′ of sensors (in the second portion 110c) and the central sensor 110a can be between two theoretical lines extending between the central sensor 110a and adjacent sensors of the first portion 110b of sensors.

In some embodiments, the first portion 110b and the second portion 110c of sensors are positioned such that at least one sensor of the second portion 110c of sensors is radially aligned with one sensor of the first portion 110b of sensors. The second group 110c″ of sensors (in the second portion 110c of sensors) may be positioned at a substantially equal second distance from the central sensor 110a (or central region 107) and substantially equidistance from adjacent sensors of the second group 110c″ of sensors. The substantially equal second distance of the second group 110c″ of sensors may be greater than the substantially equal first distance of the first group 110c′ of sensors. The second group 110c″ of sensors may be radially aligned with the first portion 110b of the sensors such that the first portion 110b of sensors are between the central sensor 110a and the second group 110c″ of sensors (in the second portion 110c of sensors). For example, a theoretical line extending between a sensor of the second group 110c″ of sensors and the central sensor 110a may also extend through a sensor of the first portion 110b of sensors. In some embodiments, the first portion 110b and the second portion 110c of sensors are positioned such that at least one sensor (e.g., the first group 110c′) of the second portion 110c of sensors is positioned circumferentially between two sensors of the first portion 110b of sensors and at least one sensor (e.g., the second group 110c″) of the second portion 110c of sensors is radially aligned with one sensor of the first portion 110b of sensors. The first group 110c′ of sensors and the second group 110c″ of sensors may be alternated in the second portion 110c of sensors such that each sensor in the first group 110c′ of sensors is circumferentially between two sensors of the second group 110c″ of sensors and each sensor in the second group 110c″ of sensors is circumferentially between two sensors of the first group 110c′ of sensors.

In some embodiments, the second portion 110c of sensors includes more sensors than the first portion 110b of sensors. More particularly, the second portion 110c of sensors include double the number of sensors in the first portion 110b of sensors. For example, the number of sensors in the first group 110c′ of sensors in second portion 110c of sensors may be equal to the number of sensors in the first portion 100b of the sensors. In some embodiments, the number of sensors in the second group 110c″ of sensors in second portion 110c of sensors may be equal to the number of sensors in the first portion 100b of the sensors. In some embodiments, the number of sensors in each of the first group 110c′ of sensors and the second group 110c″ of sensors in second portion 110c of sensors may be equal to the number of sensors in the first portion 100b of the sensors. For example, the first portion 110b of sensors may include five sensors, the first group 110c′ of the second portion 110c of sensors may include five sensors, and the second group 110c″ of the second portion 110c of sensors may include five sensors. In other embodiments, the second portion 110c of sensors may include a number of sensors that is different than the first portion 110b of sensors. For example, the second portion 110c of the sensors may include more than two times the number of sensors in the first portion 110b of sensors or less than two times the number of sensors in the first portion 110b of sensors. In some embodiments, one of the first group 110c′ or the second group 110c″ of sensors may be absent from the second group 110c″ of sensors such than the second group 110c″ of sensors includes an equal number of sensors to the first group 110c′ of sensors. In FIG. 1, the predetermined arrangement 105 includes ten sensors in the second portion 110c positioned in an array around the first portion 110b of sensors. In other embodiments, the first portion 110b and the second portion 110c of sensors positioned in an array around the central sensor 110a may include other numbers of sensors. Though not shown in FIG. 1, other embodiments, may include additional arrays of sensors radially arranged around the second portion 110c of sensors.

The plurality of sensors 110 may be positioned on the garment 100 in predetermined positions such that each sensor of the plurality of sensors 110 is a selected distance from a nearest sensor of the plurality of sensors 110. For example, each sensor of the plurality of sensors may be about less than about 7.6 cm, less than about 5.1 cm, less than about 2.5 cm, less than about 1.3 cm, greater than about 1.3 cm, greater than about 2.5 cm, greater than about 5.1 cm, greater than about 7.6 cm, about 1.3 cm to about 2.5 cm, about 2.5 cm to about 5.1 cm, about 2.5 cm to about 3.8 cm, about 3.8 cm to about 5.1 cm, about 5.1 cm to about 7.6 cm, about 5.1 cm to about 6.4 cm, about 6.4 cm to about 7.6 cm, about 1.3 cm, about 2.5 cm, about 3.8 cm, about 5.1 cm, about 6.4 cm, or about 7.6 cm from a nearest sensor of the plurality of sensors 110. Each sensor of the plurality of sensors also may be angled at a different orientation than the nearest sensor.

In some embodiments, at least some sensors of the plurality of sensors 110 may vary in length according to selecting positioning of the sensor(s) within the predetermined arrangement 105. For example, the at least some (e.g., all) sensors of the plurality of sensors include strain sensors such that the length of the sensor effects the sensitivity of the sensor. The predetermined arrangement 105, then, may include: (1) relatively longer sensors of the plurality of sensors 110 positioned in the predetermined arrangement 105 to be over regions of the abdomen where large deformations are anticipated due to fetal kicks, punches, or rolls, such as positioned to be over the lower part of the abdomen; and (2) relatively shorter sensors of the plurality of sensors 110 positioned in the predetermined arrangement 105 to be over regions of the abdomen where small deformations are anticipated, such as near the ribcage.

In some embodiments, the first group 110c′ of the second portion 110c of sensors may have a length that is shorter than the second group 110c″ of the second portion 110c of sensors. For example, the top middle sensor in FIG. 1 is positioned in the predetermined arrangement 105 on the garment 100 to be proximate to the rib cage and may be oriented in a horizontal arch and/or may be shorter than at least some sensors of the plurality of sensors 110 because less deformation is anticipated at this sensor. The first portion of sensors 110b may have a length that is uniform and/or greater than at least the first group 110c′ of the second portion 110c of sensors, as higher deformations may be expected in this position. In some embodiments, the central sensor 110a also may be have a length that is longer than at least the first group 110c′ of the second portion 110c of sensors.

The length of the sensors in the plurality of sensors 110 in the predetermined arrangement 105 also may be selected for a certain time or period of the pregnancy. For example, earlier in the pregnancy (e.g., first trimester or first half of the pregnancy), substantially all deformations are relatively small and the garment 100 may include sensors in the plurality of sensors 110 having substantially equal lengths and may be relatively short. Later in the pregnancy (e.g., third trimester or second half the pregnancy) substantially all deformations are relatively larger and the garment 100 may include sensors in the plurality of sensors 110 having substantially equal lengths and may be relatively long.

Short or relatively shorter sensors of the plurality of sensors 110 may have a length of about 0.04 mm to about 10 cm, about 0.04 cm to about 1 cm, about 1 cm to about 2 cm, about 2 cm to about 3 cm, about 3 cm to about 4 cm, about 4 cm to about 5 cm, about 5 cm to about 6 cm, about 6 cm to about 7 cm, about 7 cm to about 8 cm, about 8 cm to about 9 cm, about 9 cm to about 10 cm, less than about 10 cm, less than about 9 cm, less than about 8 cm, less than about 7 cm, less than about 6 cm, less than about 5 cm, less than about 4 cm, less than about 3 cm, less than about 2 cm, less than about 1 cm, less than about 0.75 cm, less than about 0.5 cm, or less than about 0.1 cm. Long or longer of the plurality of sensors 110 may have a length of about 10 cm to about 20 cm, about 10 cm to about 15 cm, about 15 cm to about 20 cm, about 10 cm to about 12 cm, about 12 cm to about 14 cm, about 14 cm to about 16 cm, about 16 cm to about 18 cm, about 18 cm to about 20 cm, at least about 10 cm, at least about 11 cm, at least about 12 cm, at least about 13 cm, at least about 14 cm, at least about 15 cm, at least about 16 cm, at least about 17 cm, at least about 18 cm, at least about 19 cm, or at least about 20 cm.

The garment 100 also may include conductive wire 120 connected to each sensor of the plurality of sensors 110. The conductive wire 120 also may be connected to a power source 130 such as one or more batteries and at least one of a communication module 140 configured to communicate with one or more controllers or a memory/storage device. For example, readings and/or data from one or more of the plurality of sensors 110 may be transmitted via BLUETOOTH or other wireless communication to a nearby electronic device, such as a smartphone, tablet computer, laptop computer, or desktop computer. In some embodiments, at least one (e.g., both) of the power source 130 and/or communication module 140 are secured to the garment 100. In some embodiments, at least one (e.g., both) of the power source 130 and/or the communication module 140 are detachably connected to the conductive wire 120 and/or the plurality of sensors 110. For example, at least one (e.g., both) of the power source 130 and/or the communication module 140 may be selectively disconnected from the conductive wire 120 and/or the plurality of sensors 110 (for example, to launder the garment 100) and selectively reconnected to the conductive wire 120 and/or the plurality of sensors 110. At least one (e.g., both) of the power source 130 and/or the communication module 140 may be carried in a pocket on the garment 110 or other clothes (e.g., pants) worn by the user.

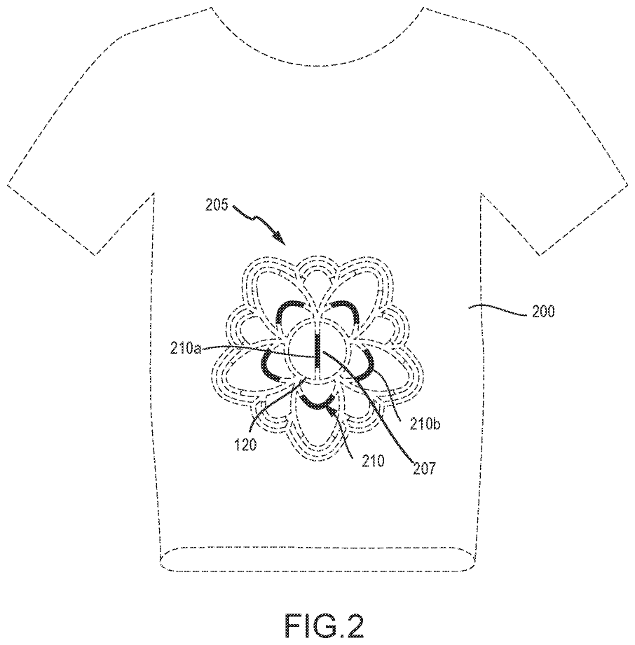

FIG. 2 is a front view of a garment 200 including a plurality of sensors 210 positioned in a predetermined arrangement 205 includes at least a central sensor 210a of the plurality of sensors 210 positioned in a central region 207 of the predetermined arrangement 205 and multiple sensors of the plurality of sensors 210 radially arranged around the central sensor 210a. Unless otherwise noted, the garment 200 and the plurality of sensors 210 may include any aspect of the garment 100, the plurality of sensors 110, and the conductive wire 120 described above. For example, the plurality of sensors 210 may include any material and/or positioning aspects of the plurality of sensors 110 described above in relation to the garment 100. Although not shown in FIG. 2, the garment 200 may include the power source 130 and the communication module 140.

In some embodiments, the plurality of sensors 210 include the central sensor 110a and a portion 210b of sensors radially arranged around the central sensor 210a. The portion 210b may include any aspect of the first portion 110b of sensor in the predetermined arrangement 105. In the garment 200, the portion 210b of the sensors includes five sensors positioned in an array around the central sensor 210a. In other embodiments, the portion 210b of sensors positioned in an array around the central sensor 110a may include other numbers of sensors. As shown in FIG. 2, some embodiments may include only a first array (e.g., first portion 210b) of sensors of the plurality of sensors 210 arranged around the central sensor 110a, and additional arrays around the first array of the sensors are absent from the garment 200. The plurality of sensors 210, then, may include at least one central sensor 210a and all the remaining sensors of the plurality of sensors 210 are positioned radially around the central sensor 210a at a substantially equal distance from the central region 207 of the predetermined arrangement 205. Moreover, the plurality of sensors 210 may include at least one central sensor 210a and all the remaining sensors of the plurality of sensors 210 may be angled at equal angles between adjacent sensors relative to the central region 207.

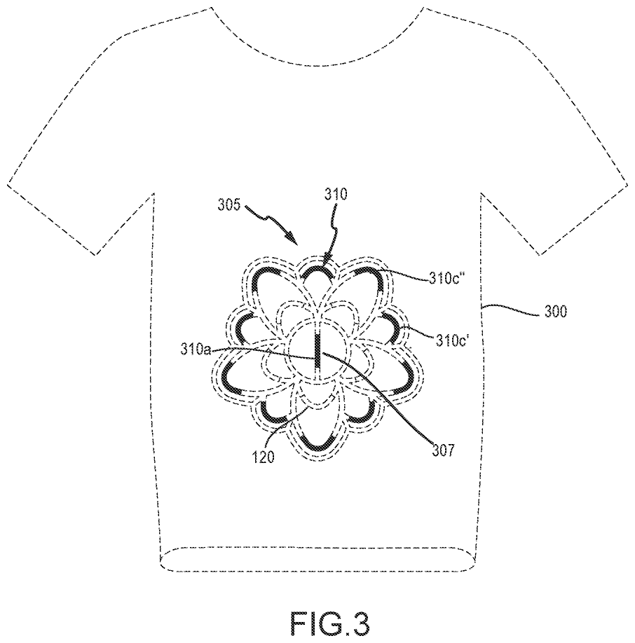

FIG. 3 is a front view of a garment 300 including a plurality of sensors 310 positioned in a predetermined arrangement 305 includes at least a central sensor 310a of the plurality of sensors 310 positioned in a central region 307 of the predetermined arrangement 305 and multiple sensors of the plurality of sensors 310 radially arranged around the central sensor 310a. Unless otherwise noted, the garment 300 and the plurality of sensors 310 may include any aspect of the garments 100, 200, the plurality of sensors 110, 210, and/or the conductive wire 120 described above. For example, the plurality of sensors 310 may include any material and/or positioning aspects of the plurality of sensors 110, 210 described above in relation to the garments 100, 200. Although not shown in FIG. 3, the garment 300 may include the power source 130 and the communication module 140.

In some embodiments of the predetermined arrangement 305, the first portion 110b, 210b of sensors in the predetermined arrangements 105, 205 is absent. The multiple sensors of the predetermined arrangement 305 may include aspect of the second portion 110c of sensors of the predetermined arrangement 305. In some embodiments, the multiple sensors of the plurality of sensors 310 radially arranged around the central sensor 310a includes a first group 310c′ of sensors and a second group 310c″ of sensors radially arranged around the central sensor 310a. The first group 310 c′ of sensors may be positioned at a substantially equal first distance from the central sensor 310a (or central region 307) and/or substantially equidistance from adjacent sensors of the first group 310c′ of sensors. The second group 310c″ of sensors may be positioned at a substantially equal second distance from the central sensor 310a (or central region 307) and/or substantially equidistance from adjacent sensors of the second group 310c″ of sensors. The substantially equal second distance of the second group 310c″ may be greater than the substantially equal first distance of the first group 310c′ of sensors. The first group 310c′ of sensors and the second group 310c″ of sensors may be alternated in the multiple sensors such that each sensor in the first group 310c′ of sensors is circumferentially between two sensors of the second group 310c″ of sensors and each sensor in the second group 310c″ of sensors is circumferentially between two sensors of the first group 310c′ of sensors. In some embodiments, the first group 310c′ may be angled at equal angles between adjacent sensors of the first group 310c′ of sensors relative to the central region 307. In some embodiments, the second group 310c′ may be angled at equal angles between adjacent sensors of the first group 310c′ of sensors relative to the central region 307. In some embodiments, each sensor in the first group 310c′ of sensors and the second group 310c″ of sensors may be angled at equal angles between adjacent sensors relative to the central region 307. In some embodiments, sensors of the plurality of sensors 310 are absent between (1) the central sensor 310a and the first group 310c′ of sensors and (2) the central sensor 310a and the second group 310c″ of sensors.

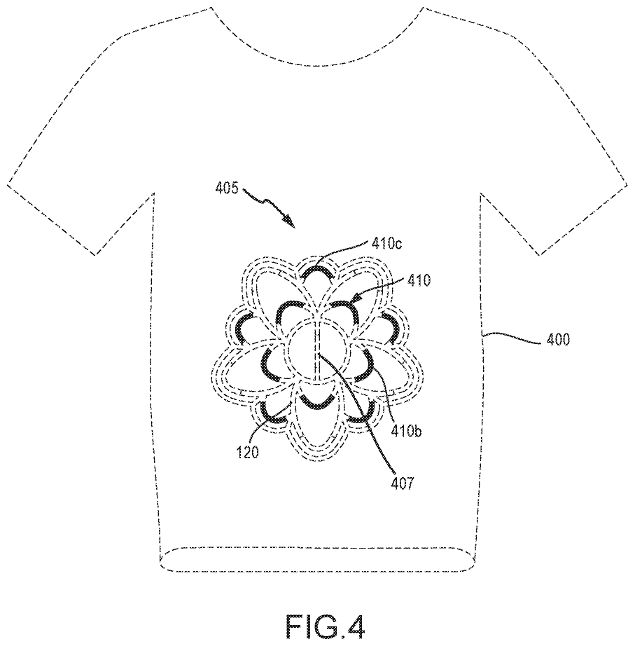

FIG. 4 is a front view of a garment 400 including a plurality of sensors 410 in a predetermined arrangement 405 having no central sensor proximate to the central region 407 of the predetermined arrangement. Unless otherwise noted, the garment 400 and the plurality of sensors 410 may include any aspect of the garments 100, 200, 300, the plurality of sensors 110, 210, 310, and/or the conductive wire 120 described above. For example, the plurality of sensors 410 may include any material and/or positioning aspects of the plurality of sensors 110, 210, 310 described above in relation to the garments 100, 200, 300. Although not shown in FIG. 4, the garment 400 may include the power source 130 and the communication module 140.

In some embodiments, the plurality of sensors 410 include multiple sensors radially arranged around the central region 407. The multiple sensors radially arranged around the central region may include a first portion 410b of sensors positioned in a first array around the central region 407 and a second portion 410c of sensors positioned in a second array around the first portion 410b of sensors (and the central region 407). In some embodiments, the first portion 410b of sensors of the plurality of sensors 410 may be radially arranged around the central region 407 at one or more first distances and the second portion 410c of sensors of the plurality of sensors 410 may be radially arranged around the central region at one or more second distances greater than the one or more first distances.

The first portion 410b of sensors in the plurality of sensors 410 may include any aspect of the first portion of sensors 110b in the predetermined arrangement 105. For example, the first portion 410b of sensors may be radially arranged around the central region 407 and/or positioned equidistance from adjacent sensors of the first portion 410b of sensors. In some embodiments, the second portion 410c of sensors in the plurality of sensors 410 may include any aspect of the first group 110c′ of sensors in the predetermined arrangement. For example, the second portion 410c of sensors may be arranged around the first portion 410b of sensors such that at least one sensor of the second portion 410c of sensors is positioned circumferentially between two sensors of the first portion 410b of sensors. Additional sensors (e.g., sensors positioned similar to the second group 110c″ of sensors in the predetermined arrangement 105) may be absent from the predetermined arrangement 405. In some embodiments (not shown), the second portion 410c of sensors shown in the predetermined arrangement 405 are absent, and the predetermined arrangement 405 instead include a second portion of sensors positioned relative to the first portion 410b similar to the positioning of the second group 110c″ of sensors relative to the first portion 110b of sensors in the predetermined arrangement 105.

In some embodiments (not shown), the predetermined arrangement 405 includes an arrangement of the plurality of sensors 410 similar to the predetermined arrangement 105 but with the central sensor 110a absent. For example, the predetermined arrangement may include the first portion 410b of sensors 410b as described above and a second portion 410c of sensors. The second portion 410c of sensors may include a first group of sensors and a second group of sensors. The first group of the second portion 410c of sensors may be positioned at a substantially equal first distance from the central region 407 and substantially equidistance from adjacent sensors of the first group of the second portion 410c of sensors. Each sensor of the first group of the second portion 410c sensors may be positioned circumferentially between two sensors of the first portion of sensors. The second group of the second portion 410c of sensors may be positioned at a substantially equal second distance from the central region 407 and substantially equidistance from adjacent sensors of the second group of the second portion 410c of sensors, with the substantially equal second distance being greater than the substantially equal first distance. The second group of the second portion 410c of sensors may be radially aligned with the first portion 410b of the sensors such that the first portion 410b of sensors are between the central region 407 and the second group of the second portion 410c sensors. The first group of sensors and the second group of sensors in the second portion 410c may be alternated in the second portion 410c of sensors such that each sensor in the first group of sensors is circumferentially between two sensors of the second group of sensors and each sensor in the second group of sensors is circumferentially between two sensors of the first group of sensors.

In the garment 400, the first portion 410b of sensors includes five sensors positioned in an array around the central region. The second portion 410c of sensors includes five sensors positioned in an array around the first portion 410b of sensors. In other embodiments, the first portion 410b and the second portion 410c of sensors positioned in an array around the central region may include other numbers of sensors. Though not shown in FIG. 4, other embodiments, may include additional arrays of sensors radially arranged around the second portion 410c of sensors. Moreover, a central sensor is absent from the plurality of sensors 410 in the garment 400, other embodiments of the garment 400 may include the central sensor. In some embodiments, the first portion 410b and the second portion 410c of sensors are positioned such that at least one sensor of the second portion 410c of sensors is positioned circumferentially between two sensors of the first portion 410b of sensors or radially spaced and between two adjacent sensors of the first portion 410b of sensors. For example, a theoretical line extending between a sensor of the second portion 410c of sensors and the central region can be between two theoretical lines extending between the central region and adjacent sensors of the first portion 410b of sensors.

In some embodiments, a system for monitoring fetal movements includes a garment and a controller operably coupled to the plurality of sensors on the garment. The garment may include any of the garments 100, 200, 300, 400 and the plurality of sensors may include any of the plurality of sensors 110, 210, 310, 410 described above. Movement or strain sensed by any sensor of the plurality of sensors is communicated to the controller. In some embodiments, the controller may include a smartphone, table computer, laptop computer, and/or desktop computer of the pregnant woman or other caretaker. Each of the plurality of sensors may measure a different strain value for a particular movement of the fetus or the pregnant woman, and these different strain values may be communicated to the controller. Using the different strain values from each sensor of the plurality of sensors, the controller is configured to determine and/or distinguish the sensed strain as a fetal movement or a movement of the pregnant woman. For example, if multiple sensors, but not all sensors, record a strain, the controller may determine that the strain was likely caused by a fetal kick from the fetus. Using the different strain values from each sensor of the plurality of sensors, the controller may be able to detect and/or distinguish fetal kicks, fetal flutters, fetal swishes, fetal rolls, and/or fetal seizures by the in utero fetus when the pregnant women is wearing the garment. The controller also may be able to determine and/or distinguish movements of the pregnant woman wearing the garment. In some embodiments, the controller may include an integrated application on an electronic device or a cloud-based application configured to interpret the data collective from the plurality of sensors using a computational model, such as a machine-learning algorithm or statistical algorithm, to classify and distinguish sensed movements such as fetal movements, maternal movements, maternal breather, etc. The controller may be configured to coordination transmission of an alert when fetal movements (or absence of fetal movements) are interpreted to reflect fetal distress.

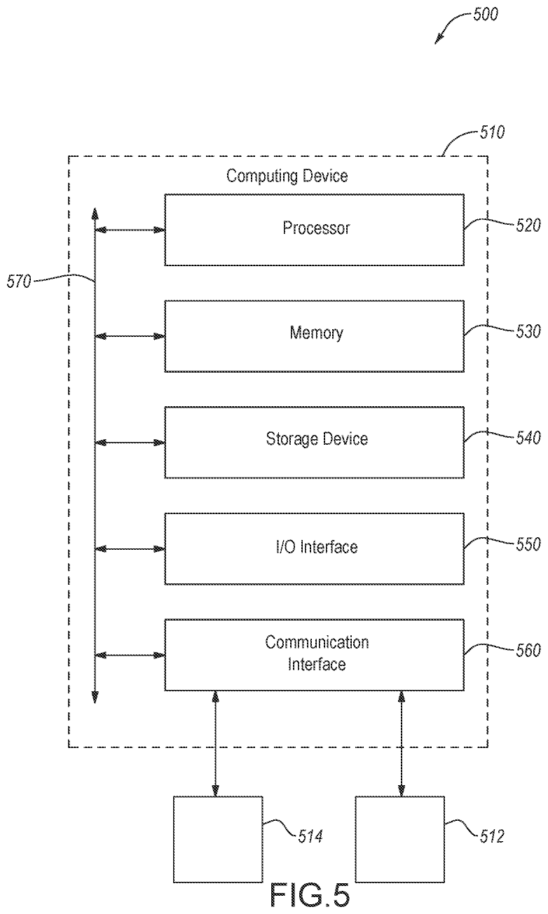

FIG. 5 is a schematic of a controller 500 for executing any of the example methods and systems disclosed herein, according to an embodiment. The controller 500 includes at least one computing device 510. The at least one computing device 510 is an exemplary computing device that may be configured to perform one or more of the acts described above. The at least one computing device 510 can include one or more servers, one or more computers (e.g., desk-top computer, lap-top computer), or one or more mobile computing devices (e.g., smartphone, tablet, etc.). The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512. In some examples, the at least one computing device 510 may include a plurality of computing devices, such as a server farm, computational network, or cluster of computing devices. Components of computing device 510 shown in FIG. 5 are described in additional detail below.

In some examples, the processor(s) 520 includes hardware for executing instructions (e.g., instructions for carrying out one or more portions of any of the methods and systems disclosed herein), such as those making up a computer program. For example, to execute instructions, the processor(s) 520 may retrieve (or fetch) the instructions from an internal register, an internal cache, the memory 530, or a storage device 540 and decode and execute them. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods disclosed herein.

In some examples, the processor 520 is configured to perform any of the acts disclosed herein or cause one or more portions of the computing device 510 or controller 500 to perform at least one of the acts disclosed herein. Such configuration can include one or more operational programs (e.g., computer program products) that are executable by the at least one processor 520.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, sensed strain from the plurality of sensors may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted access the data in the memory storage medium(s) to automatically determine a likely source of a sensed strain by the plurality of sensors.

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512, 514 or one or more networks. For example, communication interface 560 may include a network interface controller (NIC) or network adapter for communicating with an Ethernet or other wire-based network or a wireless NIC (WNIC) or wireless adapter for communicating with a wireless network, such as a WI-FI. The additional computing device 512 may include the communication module 140, and the communication module 140 may include any aspect or component of the controller 500. The additional computing device 514 may include one or more third-party electronic devices, such as emergency system, a hospital system, and other systems selected by the user.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. Computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

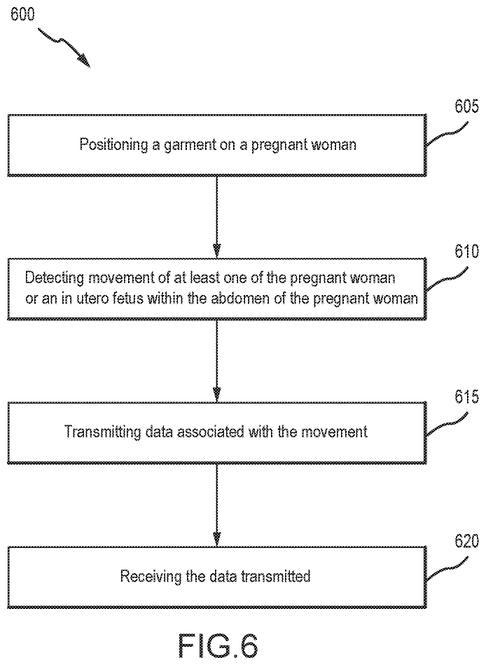

FIG. 6 is a flow diagram of a method 600 of monitoring an in utero fetus, according to an embodiment. The method 600 includes an act 605 of positioning a garment on a pregnant woman with a plurality of sensors positioned in a predetermined arrangement on the garment over the abdomen of the pregnant woman. The predetermined arrangement in the method 600 may include any predetermined arrangement 105, 205, 305, 405 described herein. In some embodiments, the predetermined arrangement of the method 600 includes a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region one or more second distances greater than the one or more first distances. The method 600 also includes an act 610 of detecting, with at least one of the plurality of sensors, movement of at least one of the pregnant women or an in utero fetus within the abdomen of the pregnant women. The method 600 also includes an act 615 of transmitting, with a communication module at least proximate to the garment, data associated with the movement detected by the at least one of the plurality of sensors. The method 600 also includes an act 620 of receiving, with a controller, the data transmitted by the communication module. Acts 605, 610, 615 and 620 of the method 600 are for illustrative purposes. For example, the acts 605, 610, 615 and 620 of the method 600 may be performed in different orders, split into multiple acts, modified, supplemented, or combined.

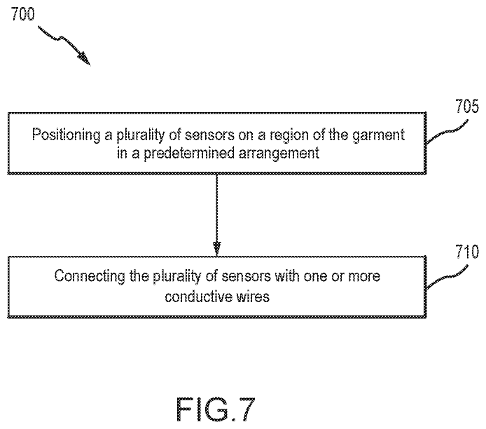

FIG. 7 is a flow diagram of a method 700 of forming a garment for a fetal monitoring system, according to an embodiment. The method 700 may include an act 705 of positioning a plurality of sensors on a region of the garment in a predetermined arrangement to be over that abdomen of a pregnant woman when worn by the pregnant woman. The plurality of sensors may be configured to sense or detect movement of at least one of the pregnant women or an in utero fetus within the abdomen of the pregnant women. The method also includes an act 710 of connecting the plurality of sensors with one or more conductive wires. Acts 705 and 710 and other acts of the method 700 are for illustrative purposes. For example, the acts of the method 700 may be performed in different orders, split into multiple acts, modified, supplemented, or combined.

The predetermined arrangement of the method 700 may include any of the predetermined arrangements 105, 205, 305, 405 described herein. In some embodiments, the act 705 of positioning the plurality of sensors on the region of the garment in the predetermined arrangement includes screen printing a composition on the region in the predetermined arrangement to form a plurality of strain gauges on the region of the garment in the predetermined arrangement. In some embodiments, the method 700 may include an act of mixing screen printing ink with metal nano-particles to form the composition before screen printing the composition on the region of the garment in the predetermined arrangement. The metal nano-particles includes one or more of nickel coated carbon fibers or nickel nanostrands.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

Features from any of the disclosed embodiments may be used in combination with one another, without limitation. In addition, other features and advantages of the present disclosure will become apparent to those of ordinary skill in the art through consideration of the following detailed description and the accompanying drawings.

While various aspects and embodiments have been disclosed herein, other aspects and embodiments are contemplated. The various aspects and embodiment disclosed herein are for purposes of illustration and are not intended to be limiting.

1. A fetal monitoring system, comprising:
a garment configured to be worn by a pregnant woman;
a plurality of sensors positioned or positionable in a predetermined arrangement on the garment to be over the abdomen of the pregnant woman, the plurality of sensors including a plurality of strain gauges configured to sense strain and detect movement of at least one of the pregnant woman or an in utero fetus within the abdomen of the pregnant woman, wherein the predetermined arrangement includes a central region and at least one of:
a central sensor of the plurality of sensors positioned in the central region of the predetermined arrangement and multiple sensors of the plurality of sensors radially arranged around the central sensor; or
a first portion of sensors of the plurality of sensors radially arranged around the central region at one or more first distances and a second portion of sensors of the plurality of sensors radially arranged around the central region at one or more second distances greater than the one or more first distances;
one or more conductive wires connecting each sensor of the plurality of sensors;
a communication module connected to at least one of the one or more conductive wires and configured to transmit data from the plurality of sensors; and
a controller configured communicate with the communication module, receive the data from the plurality of sensors transmitted by the communication module, and determine and/or distinguish strain sensed by one or more of the plurality of sensors as movement of the pregnant woman or movement of the in utero fetus.

2. The fetal monitoring system of claim 1, wherein the predetermined arrangement includes at least the central sensor of the plurality of sensors positioned in the central region of the predetermined arrangement and the multiple sensors of the plurality of sensors radially arranged around the central sensor.

3. The fetal monitoring system of claim 2, wherein the central sensor is generally straight and the multiple sensors are generally arched and concave relative to the central region.

4. The fetal monitoring system of claim 2, wherein:
the multiple sensors include at least a first portion of the multiple sensors radially arranged around the central sensor and positioned equidistance from adjacent sensors of the first portion of sensors;
the multiple sensors include the first portion of the multiple sensors radially arranged around the central sensor at one or more first distances and a second portion of sensors of the multiple sensors radially arranged around the central sensor at one or more second distances greater than the one or more first distances;
the second portion of sensors are arranged around the first portion of sensors such that at least one sensor of the second portion of sensors is positioned circumferentially between two sensors of the first portion of sensors; and
the second portion of sensors includes:
a first group of sensors positioned at a substantially equal first distance from the central sensor and substantially equidistance from adjacent sensors of the first group of sensors, each sensor of the first group of sensors being positioned circumferentially between two sensors of the first portion of sensors; and
a second group of sensors positioned at a substantially equal second distance from the central sensor and substantially equidistance from adjacent sensors of the second group of sensors, the substantially equal second distance being greater than the substantially equal first distance and the second group of sensors being radially aligned with the first portion of the sensors such that the first portion of sensors are between the central sensor and the second group of sensors,
wherein the first group of sensors and the second group of sensors are alternated in the second portion of sensors such that each sensor in the first group of sensors is circumferentially between two sensors of the second group of sensors and each sensor in the second group of sensors is circumferentially between two sensors of the first group of sensors.

5. (canceled)

6. (canceled)

7. (canceled)

8. The fetal monitoring system of claim 2, wherein the multiple sensors include:
a first group of sensors positioned at a substantially equal first distance from the central sensor and substantially equidistance from adjacent sensors of the first group of sensors; and
a second group of sensors positioned at a substantially equal second distance from the central sensor and substantially equidistance from adjacent sensors of the second group of sensors, the substantially equal second distance being greater than the substantially equal first distance;
wherein the first group of sensors and the second group of sensors are alternated in the multiple sensors such that each sensor in the first group of sensors is circumferentially between two sensors of the second group of sensors and each sensor in the second group of sensors is circumferentially between two sensors of the first group of sensors;
wherein sensors of the plurality of sensors are absent between the multiple sensors and the central sensor; and
wherein each sensor of the first group of sensors has a length that is less than a length of each sensor of the second group of sensors.

9. (canceled)

10. (canceled)

11. The fetal monitoring system of claim 1, wherein:
the central sensor is absent from the predetermined arrangement of the plurality of sensors and the arrangement of the plurality of sensors includes the first portion of sensors of the plurality of sensors radially arranged around the central region at one or more first distances and the second portion of sensors of the plurality of sensors radially arranged around the central region at one or more second distances greater than the one or more first distances;
the plurality of sensors are generally arched and concave relative to the central region;
the first portion of sensors are radially arranged around the central region and positioned equidistance from adjacent sensors of the first portion of sensors;
the second portion of sensors are arranged around the first portion of sensors such that at least one sensor of the second portion of sensors is positioned circumferentially between two sensors of the first portion of sensors, wherein the second portion of sensors includes:
a first group of sensors positioned at a substantially equal first distance from the central region and substantially equidistance from adjacent sensors of the first group of sensors, each sensor of the first group of sensors being positioned circumferentially between two sensors of the first portion of sensors; and
a second group of sensors positioned at a substantially equal second distance from the central region and substantially equidistance from adjacent sensors of the second group of sensors, the substantially equal second distance being greater than the substantially equal first distance and the second group of sensors being radially aligned with the first portion of the sensors such that the first portion of sensors are between the central region and the second group of sensors,
wherein the first group of sensors and the second group of sensors are alternated in the second portion of sensors such that each sensor in the first group of sensors is circumferentially between two sensors of the second group of sensors and each sensor in the second group of sensors is circumferentially between two sensors of the first group of sensors, and
wherein each sensor of the first group of sensors has a length that is less than a length of each sensors of the second group of sensors.

12. (canceled)

13. (canceled)

14. (canceled)

15. (canceled)

16. (canceled)

17. (canceled)

18. The fetal monitoring system of claim 1, wherein the plurality of strain gauges are screen printed onto the garment and include a composite material including screen printing ink blended with metal nano-particles, the metal nano-particles including at least nickel coated carbon fibers.

19. The fetal monitoring system of claim 18, wherein the metal nano-particles includes nickel coated carbon fibers and nickel nanostrands, and wherein the composite material includes about 5% to about 25% saturation of the metal nanoparticles.

20. A garment for use in a fetal monitoring system, the garment comprising:
a region positioned to cover the abdomen of a pregnant woman; and
a plurality of sensors positioned or positionable in a predetermined arrangement on the region of the garment to be over the abdomen of the pregnant woman and configured to sense or detect movement of at least one of the pregnant woman or an in utero fetus within the abdomen of the pregnant woman, wherein the predetermined arrangement includes a central region and at least one of:
a central sensor of the plurality of sensors positioned in the central region of the predetermined arrangement and multiple sensors of the plurality of sensors radially arranged around the central sensor; or
a first portion of sensors of the plurality of sensors radially arranged around the central region at one or more first distances and a second portion of sensors of the plurality of sensors radially arranged around the central region at one or more second distances greater than the one or more first distances;
wherein the plurality of sensors include a plurality of strain gauges secured to the the garment and configured to sense strain, the strain gauges including a composite material of ink blended with metal nano-particles and having about 5% to about 25% saturation of the metal nano-particles.

21. The garment of claim 20, further comprising one or more conductive wires connecting each sensor of the plurality of sensors.

22. The garment of claim 20, wherein the predetermined arrangement includes at least the central sensor of the plurality of sensors positioned in the central region of the predetermined arrangement and the multiple sensors of the plurality of sensors radially arranged around the central sensor.

23. The garment of claim 22, wherein the central sensor is generally straight and the multiple sensors are generally arched and concave relative to the central region.

24. The garment of claim 22, wherein:
the multiple sensors include at least a first portion of the multiple sensors radially arranged around the central sensor and positioned equidistance from adjacent sensors of the first portion of sensors;
the multiple sensors include the first portion of the multiple sensors radially arranged around the central sensor at one or more first distances and a second portion of sensors of the multiple sensors radially arranged around the central sensor at one or more second distances greater than the one or more first distances;
the second portion of sensors are arranged around the first portion of sensors such that at least one sensor of the second portion of sensors is positioned circumferentially between two sensors of the first portion of sensors, wherein the second portion of sensors includes:
a first group of sensors positioned at a substantially equal first distance from the central sensor and substantially equidistance from adjacent sensors of the first group of sensors, each sensor of the first group of sensors being positioned circumferentially between two sensors of the first portion of sensors; and
a second group of sensors positioned at a substantially equal second distance from the central sensor and substantially equidistance from adjacent sensors of the second group of sensors, the substantially equal second distance being greater than the substantially equal first distance and the second group of sensors being radially aligned with the first portion of the sensors such that the first portion of sensors are between the central sensor and the second group of sensors,
wherein the first group of sensors and the second group of sensors are alternated in the second portion of sensors such that each sensor in the first group of sensors is circumferentially between two sensors of the second group of sensors and each sensor in the second group of sensors is circumferentially between two sensors of the first group of sensors;
each sensor of the first group of sensors has a length shorter than a length of each sensor of the second group of sensors.

25. (canceled)

26. (canceled)

27. (canceled)

28. (canceled)

29. The garment of claim 22, wherein the multiple sensors include:
a first group of sensors positioned at a substantially equal first distance from the central sensor and substantially equidistance from adjacent sensors of the first group of sensors; and
a second group of sensors positioned at a substantially equal second distance from the central sensor and substantially equidistance from adjacent sensors of the second group of sensors, the substantially equal second distance being greater than the substantially equal first distance;
wherein the first group of sensors and the second group of sensors are alternated in the multiple sensors such that each sensor in the first group of sensors is circumferentially between two sensors of the second group of sensors and each sensor in the second group of sensors is circumferentially between two sensors of the first group of sensors;
wherein sensors of the plurality of sensors are absent between the multiple sensors and the central sensor; and
wherein each sensor of the first group of sensors has a length shorter than a length of each sensor of the second group of sensors.

30. (canceled)

31. (canceled)

32. The garment of claim 22, wherein:
the central sensor is absent from the predetermined arrangement of the plurality of sensors and the arrangement of the plurality of sensors includes the first portion of sensors of the plurality of sensors radially arranged around the central region at one or more first distances and the second portion of sensors of the plurality of sensors radially arranged around the central region at one or more second distances greater than the one or more first distances;
the plurality of sensors are generally arched and concave relative to the central region;
the first portion of sensors are radially arranged around the central region and positioned equidistance from adjacent sensors of the first portion of sensors;
the second portion of sensors are arranged around the first portion of sensors such that at least one sensor of the second portion of sensors is positioned circumferentially between two sensors of the first portion of sensors, wherein the second portion of sensors includes:
a first group of sensors positioned at a substantially equal first distance from the central region and substantially equidistance from adjacent sensors of the first group of sensors, each sensor of the first group of sensors being positioned circumferentially between two sensors of the first portion of sensors; and
a second group of sensors positioned at a substantially equal second distance from the central region and substantially equidistance from adjacent sensors of the second group of sensors, the substantially equal second distance being greater than the substantially equal first distance and the second group of sensors being radially aligned with the first portion of the sensors such that the first portion of sensors are between the central region and the second group of sensors,
wherein the first group of sensors and the second group of sensors are alternated in the second portion of sensors such that each sensor in the first group of sensors is circumferentially between two sensors of the second group of sensors and each sensor in the second group of sensors is circumferentially between two sensors of the first group of sensors; and
each sensor of the first group of sensors has a length shorter than a length of each sensor of the second group of sensors.

33. (canceled)

34. (canceled)

35. (canceled)

36. (canceled)

37. (canceled)

38. (canceled)

39. (canceled)

40. The garment of claim 20, wherein the metal nano-particles in the composite material includes at least nickel coated carbon fibers.

41. A method of monitoring an in utero fetus, the method comprising:
positioning a garment on a pregnant woman with a plurality of sensors positioned in a predetermined arrangement on the garment over the abdomen of the pregnant woman, wherein the predetermined arrangement includes a central region and at least one of (1) a central sensor of the plurality of sensors positioned in the central region of the predetermined arrangement and multiple sensors of the plurality of sensors radially arranged around the central sensor or (2) a first portion of sensors of the plurality of sensors radially arranged around the central region at one or more first distances and a second portion of sensors of the plurality of sensors radially arranged around the central region at one or more second distances greater than the one or more first distances, wherein the plurality of sensors include a plurality of strain gauges configured to sense strain;
detecting, with strain on at least one of the plurality of sensors, movement of at least one of the pregnant woman or an in utero fetus within the abdomen of the pregnant woman;
transmitting, with a communication module at least proximate to the garment, data associated with the movement detected by the strain on the at least one of the plurality of sensors;
receiving, with a controller, the data transmitted by the communication module; and
determining and/or distinguishing, with the controller, strain sensed by one or more of the plurality of sensors as movement of the pregnant woman or movement of the in utero fetus.

42. A method of forming a garment for a fetal monitoring system, the method comprising:
positioning a plurality of sensors on a region of the garment in a predetermined arrangement to be over an abdomen of a pregnant woman when worn by the pregnant woman, the plurality of sensors including a plurality of strain gauges configured to sense strain and including a composite material of ink blended with metal nano-particles and having about 5% to about 25% saturation of the metal nano-particles, the plurality of sensors configured to sense or detect movement of at least one of the pregnant woman or an in utero fetus within the abdomen of the pregnant woman, wherein the predetermined arrangement includes a central region and at least one of:
a central sensor of the plurality of sensors positioned in the central region of the predetermined arrangement and multiple sensors of the plurality of sensors radially arranged around the central sensor; or
a first portion of sensors of the plurality of sensors radially arranged around the central region at one or more first distances and a second portion of sensors of the plurality of sensors radially arranged around the central region at one or more second distances greater than the one or more first distances; and
connecting the plurality of sensors with one or more conductive wires.

43. The method of claim 42, wherein positioning the plurality of sensors on the region of the garment in the predetermined arrangement includes screen printing the composite material on the region in the predetermined arrangement to form a plurality of strain gauges on the region of the garment in the predetermined arrangement.

44. The method of claim 42, further comprising mixing screen printing ink with metal nano-particles to form the composite material, the composite material including at least nickel coated carbon fibers.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The method of claim 42, further comprising securing the one or more conductive wires to the garment.

60. The method of claim 59, further comprising connecting the one or more conductive wires to a power source and a communication module.

61. The fetal monitoring system of claim 1, wherein, responsive to strain sensed by one or more of the plurality of sensors, the controller is configured to detect and/or distinguish between fetal kicks, fetal flutters, fetal swishes, fetal rolls, and/or fetal seizures by the in utero fetus.

62. The garment of claim 20, wherein the plurality of sensors are screen printed onto the garment and have a thickness of about 0.1 mm to about 1.0 mm.

63. The method of claim 41, further comprising detecting and/or distinguishing, with the controller, between fetal kicks, fetal flutters, fetal swishes, fetal rolls, and/or fetal seizures by the in utero fetus.
